(19) United States
(12) Patent Application Publication
(10) Pub. No.: US 2005/0119631 A1
(43) Pub. Date: Jun. 2, 2005

(54) Method of producing absorptive areas for protective products

(75) Inventors: Tamar Giloh, Radcliffe Manchester (GB); Ehud Giloh, Radcliffe Manchester (GB); Yoav Nir, Radcliffe Manchester (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Tamicare Ltd.

(21) Appl. No.: 10/970,949

(22) Filed: Oct. 22, 2004

Related U.S. Application Data
(60) Provisional application No. 60/513,902, filed on Oct. 22, 2003.

(51) Int. Cl. A61F 13/15; A61F 13/20

(52) U.S. Cl. 604/367

(57) ABSTRACT

The invention provides an absorptive article that comprises side-by-side dedicated functional zones that improve flow of fluids to retention and/or absorptive areas, thereby efficiently collecting fluids from an individual or other source of fluids without leakage. In addition the invention may further provide functional zones that release material such as an antibiotic or anti-fungal agent towards the individual or object.

This application claims the benefit of priority from U.S. Provisional application No. 60/513,902 filed Oct. 22, 2003, which is incorporated by reference in its entirety.

### FIELD OF THE INVENTION

The present invention relates to absorbent articles generally as well as to methods for manufacture thereof.

### BACKGROUND OF THE INVENTION

It is known and described in the art that absorbent articles, mostly for feminine hygiene and adult incontinence products, suffer from leakage problems related to insufficient distribution of body fluids from a target area to the outer areas of the absorptive device. This is mostly due to the way conventional absorption cores for absorption articles are manufactured (e.g. different materials are built as a compressed multi-layered structure with primarily even distribution throughout the whole layered area. Such a layered structure results in equal fluid wicking in all directions, so that the fluids are distributed in a circular-type pattern. Fluids distributed in such a manner tend to reach the edges of a conventional rectangular article very quickly, causing leakage. The phenomena prevents the utilization of the remote areas of the article and limits the efficiency of the article's total length. In addition, the phenomena called “gel block” caused by super absorbent material further limits article performance.

The invention provides an improved absorbent article that solves these problems. Specifically, the invention provides articles of dedicated ultrathin absorption and medical products and methods of producing such articles. The articles allow significant improvement in product performance, efficiency, and discreetness while not hindering the wearer's movability or comfort.

### SUMMARY OF THE INVENTION

The invention provides an absorptive article comprising a single layer of a plurality of defined functional zones arranged in a predetermined pattern on a surface, wherein at least one of the functional zones comprises an absorptive material. The surface and/or its support can be made of any suitable material such as natural rubber film (latex), synthetic rubber (silicone & polyurethanes, and any suitable synthetic polymer materials), nylon film, nonwoven material (polyolefin of different types), micro porous films (polyolefin of different types), and the like, as known in the art. In one aspect, the absorptive article comprises absorption particles that comprise, for example, fluff or super absorbent fibers (SAF), or a combination thereof. In other aspects, at least one of the functional zones is a wicking area; at least one of the functional zones is a zero density zone; at least one of the functional zones is a barrier area; and at least one of the functional zones is a retention area.

In some aspects, such zones can be arranged on a surface that is then associated with a substrate, such as a support. In other aspects, the zones can be arranged directly on a support.

In still other aspects, an absorptive article of the invention can be associated with a support that comprises an inner surface with a first portion having the functional zones and a second portion, wherein the second portion is formed with multiple perforations to permit passage of perspiration therethrough. In certain aspects, the support is a protective garment, a protective undergarment, a medical dressing, a nursing pad, a diaper, an absorbent pad, a cosmetic pad, or a cleansing article. An absorbent pad can further include an adhesive backing.

In yet another aspect, an absorptive article of the invention can further comprise a cover over the functional zones that can contact a source of fluid. A cover can comprise a permeable, hydrophobic material and can be applied to the article, for example, by spraying or flocking.

Specific preferred embodiments of the invention will become evident from the following more detailed description of certain preferred embodiments and the claims.

### BRIEF DESCRIPTION OF THE DRAWING

FIG. 1 shows a plan view of a crotch portion of an article of the invention. A pattern of absorptive areas is shown as described above.

FIG. 2 shows a plan view of an article of the invention (15), the article being a medical bandage comprising distribution zones (6), a target zone (7), a medical treatment capsule zone (8); diagnostic particles (9); barrier zone (10), sensing strap/fibers zone (11), retention zone (12); straps (13), and perforations (14).

FIG. 3A shows a plan view of an article of the invention (17), the article being a nursing pad, comprising distribution zones (6), a target zone (7), a medical treatment zone (8); barrier zone (10), sensing zone (11), retention zone (12) perforations (14) and 0 density zone (16).

FIG. 3B shows the liquid direction at the different zones on the article shown in FIG. 3A.

FIG. 4 shows a plan view of an article of the invention (18) the article being a diaper for baby-girl.

### DETAILED DESCRIPTION OF THE INVENTION

The invention provides an absorptive article that can be worn by an individual to prevent leakage of body fluids, including, but not limited to urine, blood, stool, plasma, menses, breast milk, and serum. An absorptive article of the invention can be, for example, a protective undergarment, a medical dressing, a nursing pad, a diaper, cosmetic pad, treatment pad, or a cleansing article.

In one embodiment, an absorptive article of the invention comprises a plurality of single layer of defined functional zones in a predetermined pattern, such as a side-by-side orientation. For example, functional zones can include various absorptive areas, retention areas, wicking areas, barrier areas, and the like strategically positioned in various patterns and sizes on a background, side-by-side, on the article. The pattern of each zone on an article can form a specific design according to a specific function for the zone and the specific article purpose. For example, wicking areas can be formed by designing a lateral root-like pattern across the surface of the article, wherein the wicking area comprises hydrophobic materials (e.g. polyester fibers and/or hydrophilic materials like fluff; retention particles can be clustered for localizing fluids; and barrier materials, such as polyethylene and polypropylene and different types of adhesives (e.g. latex adhesives) can be arranged to form barrier walls. The zones can be different heights at different locations on the surface of the article, for example by using fibers of various lengths or particles of various heights. Also, the zones can differ in density. For example, a zone with zero density (e.g. empty) can serve to control flow of fluids by surface tension along the edges of the zone. In addition, a wicking zone connected to a zero density zone can increase fluid flow speed to a desired location on the article.

In a particular embodiment, an absorptive article of the invention can be designed to provide areas of distribution in places that are most likely to contact body fluids. For example, if the article is a sanitary towel, the distribution and absorptive zones can be positioned differently depending on whether a male or female will be wearing the article. Conventional protective undergarments comprise a large pad or generally consist of a thick, absorbent material, which are uncomfortable and bulky and is limited in performance ability. In contrast, an article of the invention controls fluids distribution speed towards the desired directions and target destination retention zones, it is much more efficient, is less cumbersome and provides superior protection.

Absorptive areas can be created by lateral application of various types of particles, such as fluff; super absorbent fibers (SAF); treatment material to treat existing conditions or prevent infection (including, but not limited to antibiotics, antifungal compounds, antiviral compounds, and analgesics); diagnostic material (including, but not limited to compounds that test for chemical adulterants, pH indicators, and labeled antibodies); odor control material; liquid absorbing particulate gels; odor absorbing gels; or any other absorbent material. The absorptive areas can be designed to form any desired pattern and size in a lateral or vertical dimension relative to the article. In one embodiment, an absorptive area can comprise absorptive particles that are contained in a liquid permeable material to ensure the particle remain in a discrete location. For example, absorptive particles can be contained in a mesh container, and the container can be attached (e.g. glued) to a surface.

In one embodiment, an article of the invention can serve multiple functions based on the particular types of particles that are used to create the absorptive areas. For example, particles can be used for acquisition, distribution, or as a barrier for body fluids. In a particular embodiment, such particles can be strategically placed in patterns that cause body fluids to be distributed quickly to absorption particles, while barrier particles surrounding the absorption particles ensure that leakage does not occur.

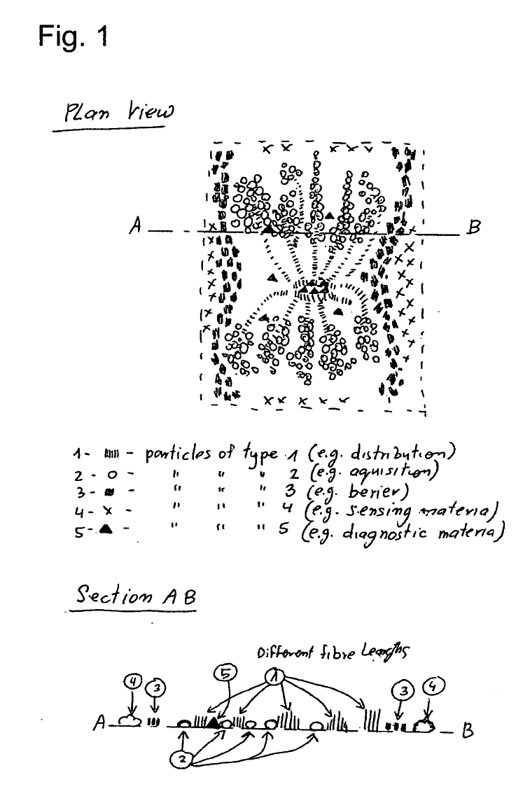

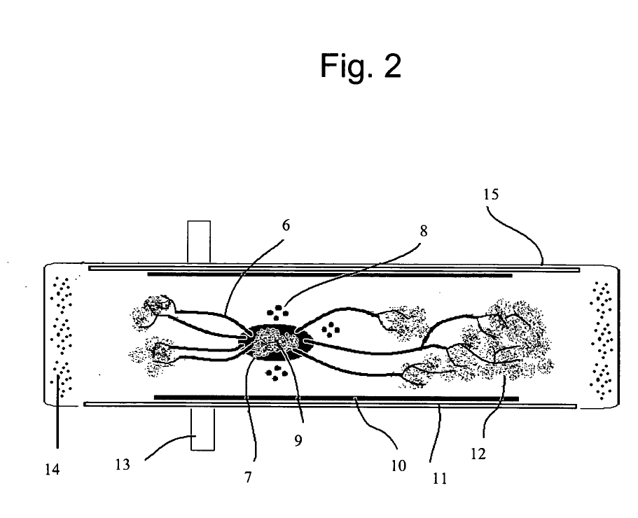

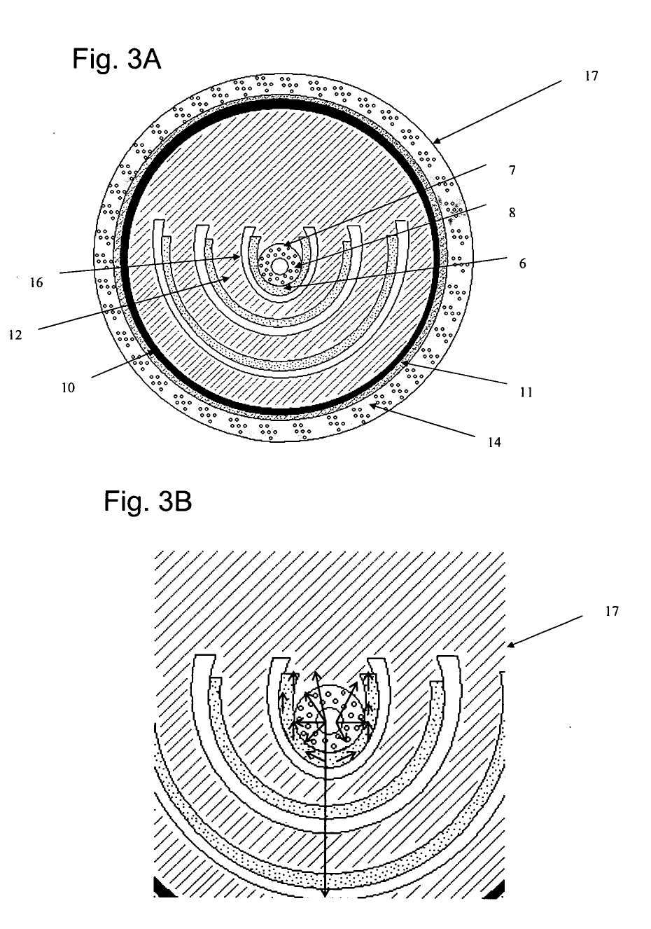

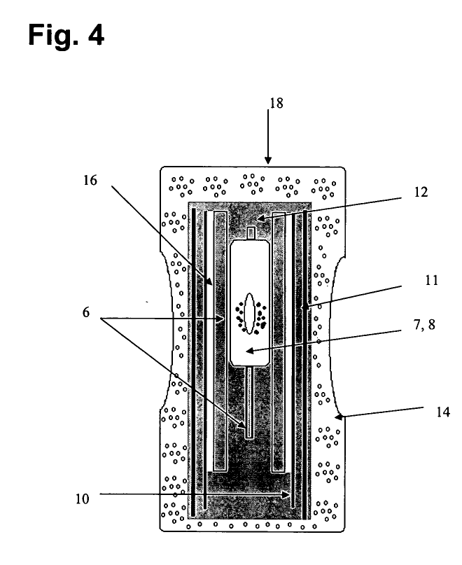

FIG. 1 shows one possible pattern of various particles used on an article of the invention. In FIG. 1, diagnostic material and distribution particles are placed primarily at the position most likely to contact body fluid. Body fluid will be distributed quickly to the acquisition particles, which are surrounded by barrier particles. A sensing material is also shown in FIG. 1 surrounding the barrier, which indicates a need for replacing the article with a fresh article of the invention. Section AB in FIG. 1 shows the side-by-side structure of the article and that the particles in the various areas can be various fiber lengths. Thus, the absorptive areas that make up the single layer on the article can be various heights.

In one embodiment, an absorptive article of the invention can be an ultra thin, protective undergarment that is generally thinner, lighter, more comfortable, and permits greater maneuverability for the wearer than conventional protective undergarments. For example, an absorptive article of the invention can be designed for feminine hygiene (e.g. an undergarment designed to absorb and prevent leakage of menses) or for infant or adult incontinence (e.g. an undergarment or diaper designed to absorb and prevent leakage of body fluids and waste products).

In other embodiments, an absorptive article of the invention can be a medical dressing, such as a bandage, that can be used to absorb body fluids excreted from a wound, including, but not limited to a surgical wound, or an absorptive article of the invention can be a nursing pad, which can be worn by nursing mothers to prevent leakage of body fluids onto clothing.

In one embodiment, an article of the invention comprises an integrally formed body formed of a liquid impermeable material comprising a single layer of absorptive areas as described above. Suitable but not limiting examples of a liquid impermeable material include a natural elastiomer, such as latex, or artificial elastiomer, such as silicone rubber or polyurethane. In one embodiment, an integrally formed body can be incorporated into the article of the invention prior to or after having a single layer of absorptive areas incorporated therein.

In yet another embodiment, the invention provides an absorptive article that comprises a first portion that comprises the absorptive areas, and a second portion that is formed with multiple perforations to permit passage of perspiration therethrough, as described herein, strategically patterned to prevent passage of body fluids therethrough. In a particular embodiment, the acquisition zone can be placed in positions most likely to contact body discharge source, thereby allowing the article to comprise a larger first portion to minimize discomfort caused by perspiration. The second portion can be the integrally formed body described above, fixedly attached to the first portion.

In still another embodiment, an article of the invention can comprise an adjustment device, such as a strap, clip, fastener, or the like, that can be integral with the article or separate from, but attached to, the article. An adjustment device can provide a means for making an article of the invention fit more snugly or comfortably on an individual. An adjustment device can also provide a means for attaching an article of the invention to or removing an article of the invention from an individual. Such connectors are described, for example, in U.S. Provisional Application Ser. No. 60/429,647, filed Nov. 27, 2002, which is hereby incorporated by reference.

In a particular embodiment, a single layer of absorptive areas can be produced by flocking. Many flocking fibers (natural and artificial) are known in the art. Flocking procedures generally involve applying such fibers directly onto a substrate that has been coated with an adhesive. In one embodiment of the invention, a flocked material can be either attached to the surface of an article of the invention while its surface is sticky, or adhered on a dry surface using adhesives. Latex based adhesives, or the like, can be sprayed or applied by brush, alternatively a double sided adhesive could be first adhered to the surface and then the particles are flocked on it.

The flocking procedure can be accomplished using special equipment that electrically charges the flock particles, which causes the particles to extend upward. For light production flocking, a Flock King Gun or CP Plus Electrostatic Flocking Unit can be used (Cellusuede Products, Rockford, Ill.). For mass production, Fabricoat Conveyor Units for 2 dimensional objects, Fabricoat Pneumatic/Electrostatic Flock System for more complicated objects, or Pattern Flocking with Rotary Screen Printer Aswell can be used (Campbell Coutts Ltd., Southampton, U.K.). Several flocking guns, each for a different particle, or single gun with different flocking containers can be used to create a pattern for an article of the invention. The pattern can be controlled by software. The resulting flocked fibers are thereby anchored to the substrate at right angles.

Flocking can also be accomplished manually by preparing a background with adhesive and mechanical masks representing a pattern for each material to be flocked. The material can then be poured manually through the mask to create the desired pattern. Alternatively, pre-prepared sheets of each material, cut according to the desired pattern, can be adhered to the surface of the article or integrally formed body. The sheets can also include double sided adhesive.

Functional zones on an article of the invention can be formed directly on a background rather than by compressing multiple layers together. The lack of compressed layers renders an article of the invention smoother, more resilient, more stretchable, and more flexible than conventional articles that comprise a core of compressed materials.

In one embodiment, an absorptive article of the invention can comprise a cover over the functional zones, wherein the cover faces the body of the article user. Thus, the cover contacts the source of the fluid that is to be absorbed by the article of the invention. For, example, the absorptive article of the invention is placed on a wound with the cover between the wound and the functional zone areas. The cover can comprise, for example, a permeable, hydrophobic material that can be sprayed, flocked, or applied by any other method as known in the art. The cover can be a bodyside liquid-permeable topsheet as known in the art (e.g. aperture polyethylene films—as produced by Tredegar Corp., South Wales, Great Britain; perforated polypropylene nonwovens—as produced by Tredegar Corp., nonwoven distribution/acquisition sheets as produced by Shalag Industries, Upper Galilee, Israel).

In another embodiment, functional zones of an article of the invention can comprise one or more microfluidics devices to further enhance fluid flow and retention in desired locations and directions. A microfluidic device can serve, for example, to accurately and efficiently control fluid distribution from the source area directly to target zones and prevent wet back by a non-retum valve. Hydrophobic microfluidic devices can be wires, straps, or bands that are made of a material such as polyethylene, can improve liquid distribution by liquid surface tension, which causes liquids to migrate over the devices. A microfluidics device can also be a capillary tube.

In yet another embodiment, each functional zone of an absorptive article of the invention can be a different color to improve aesthetic quality and/or mask the color of certain fluids that contact the article of the invention.

It will be appreciated by persons skilled in the art of that the present invention is not limited by what has been particularly shown and described hereinabove. Rather the scope of the present invention includes variations and modification of the various features described in the specification and shown in the drawings which may occur to a person of ordinary skill in the art upon reading the foregoing description and which are not in the prior art.

1. An absorptive article comprising a single layer of a plurality of defined functional zones arranged in a predetermined pattern on a surface, wherein at least one of the functional zones comprises an absorptive area.

2. The absorptive article of claim 1, wherein the absorptive area comprises absorption particles.

3. The absorptive article of claim 2, wherein the absorption particles comprise fluff or super absorbent fibers (SAF).

4. The absorptive article of claim 1, wherein at least one of the functional zones is a wicking area.

5. The absorptive article of claim 1, wherein at least one of the functional zones is a zero density zone.

6. The absorptive article of claim 1, wherein at least one of the functional zones is a barrier area.

7. The absorptive article of claim 1, wherein at least one of the functional zones is a retention area.

8. The absorptive article of claim 1, wherein the surface is associated with a support.

9. The absorptive article of claim 8, wherein the support comprises an integrally formed body formed of a liquid impermeable material having an inner surface with a first portion and a second portion, wherein the background material is associated with the inner surface of the first portion.

10. The absorptive article of claim 9, wherein the second portion is formed with multiple perforations to permit passage of perspiration therethrough.

11. The absorptive article of claim 9, wherein the liquid impermeable material is a natural elastiomer.

12. The absorptive article of claim 11, wherein the natural elastiomer is latex.

13. The absorptive article of claim 9, wherein the liquid impermeable material is a synthetic elastiomer.

14. The absorptive article of claim 13, wherein the synthetic elastiomer is silicon rubber or polyurethane.

15. The absorptive article of claim 9, wherein the liquid impermeable material is a nonwoven material.

16. The absorptive article of claim 15, wherein the liquid impermeable material is polyolefin.

17. The absorptive article of claim 9, wherein the article is a medical dressing.

18. The absorptive article of claim 9, wherein the article is a protective undergarment.

19. The absorptive article of claim 9, wherein the article is a nursing pad.

20. The absorptive article of claim 9, wherein the article is a diaper.

21. The absorptive article of claim 1 further comprising a cover over the functional zones.

22. The absorptive article of claim 21, wherein the cover comprises a permeable, hydrophobic material.

23. The absorptive article of claim 22, wherein the permeable, hydrophobic material is applied to the article by spraying or flocking.
